(19) United States
(12) Patent Application Publication
(10) Pub. No.: US 2007/0187456 A1
(43) Pub. Date: Aug. 16, 2007

(54) Angled surgical fastener apparatus

(75) Inventors: Frank J. Viola, Sandy Hook, CT (US); David C. Racenet, Litchfield, CT (US); Ernest Aranyi, Easton, CT (US); Richard D. Gresham, Guilford, CT (US); Paul A. Scirica, Huntington, CT (US); David Farascioni, Bethel, CT (US); Kenneth H. Whitfield, New Haven, CT (US); Philip C. Roy, Hamden, CT (US); Earl Zergiebel, Guilford, CT (US)

Correspondence Address: UNITED STATES SURGICAL,;A DIVISION OF TYCO HEALTHCARE GROUP LP, 195 MCDERMOTT ROAD, NORTH HAVEN, CT, 06473, US

(73) Assignee: Tyco Healthcare Group LP

(21) Appl. No.: 11/786,198

(22) Filed: Apr. 10, 2007

Related U.S. Application Data
(63) Continuation of application No. 10/528,889, filed on Mar. 23, 2005.
(60) Provisional application No. 60/416,332, filed on Oct. 4, 2002.

(51) Int. Cl. A61B 17/10

(52) U.S. Cl. 227/175.1

(57) ABSTRACT

A surgical fastening apparatus includes a handle, an elongated member extending distally from the handle and a fastener applier and anvil head disposed at the distal end of the elongated member. The elongated member defines a distal offset end portion upon which the fastener applier and the anvil head are mounted. The offset end portion is angularly oriented with respect to the axis of the instrument at a predetermined fixed angle (e.g., either 45°, 60° and 75° relative to the axis of the instrument) thereby positioning the fastener applier and the anvil head at the fixed angle. This feature facilitates positioning of the tissue with respect to the instrument and enhances visual access to the tissue when appropriately disposed within the fastener applier and the anvil head. Various mechanisms for effectuating the clamping of the tissue between the fastener holder and anvil, and the firing of the instrument are disclosed.

### CROSS REFERENCE TO RELATED APPLICATION:

This application claims priority from of U.S. Provisional Application Ser. No. 60/416,332 filed on Oct. 4, 2002 by Frank J. Viola et al. entitled “ANGLED SURGICAL FASTENER APPARATUS”, and incorporates its entire contents by reference herein.

### BACKGROUND OF THE INVENTION

1. Field of the Disclosure

The present invention relates to a surgical fastening apparatus, and more particularly, a surgical fastener apparatus having a fastener applying assembly which is offset relative to the axis of the apparatus.

2. Description of the Prior Art

Surgical fastener apparatti in which a plurality of surgical fasteners are driven substantially simultaneously or in progression from a fastener holder to produce an array of fasteners in body tissue are known. Typically, these apparatti include a fastener holder disposed on one side of the tissue to be fastened, an anvil parallel to the fastener holder on the other side of the tissue to be fastened, means for linearly translating the fastener holder and the anvil toward one another so that the tissue is clamped therebetween, and means for driving the fasteners from the fastener holder so that the ends of the fasteners pass through the tissue and are crimped against the anvil, thereby producing an array of finished fasteners in the tissue.

Such fastener applying apparatti may include instruments adapted to drive metallic staples through the tissue. The staples are clinched by the anvil to be secured in the tissue. Alternatively, the fastener applying apparatus may be adapted for use with a two-part fastener, i.e., a fastener part disposed in the fastener holder component of the assembly and a retainer part disposed in the anvil component of the apparatus. The legs of the fastener parts are driven through the tissue to interlock with the retainer parts to secure the fasteners in the tissue. Such surgical fasteners may be absorbable or non-absorbable.

Instruments of the type described above are available for performing several different types of surgical fastener procedures and may require fastener or staple arrays of various configurations. An illustrative type of these instruments is the thoracic-abdominal surgical fastener instrument. This instrument is typically used for forming a linear row of fasteners laterally through hollow body organs such as the thorax, trachea, stomach, or intestines. Fastener instruments of this type generally have a U-shaped structure at the distal end of the instrument. The U-shaped structure is positioned around the tissue to be stapled. The anvil is a component on the distal leg of the U-shaped structure while the fastener holder is mounted relative to the proximal leg of the structure. An actuator at the handle of the instrument provides relative movement of the fastener holder and the anvil to clamp the tissue between the opposing faces of the anvil and the fastener holder. A firing lever is thereafter operated to simultaneously drive all of the fasteners out of the fastener holder through the tissue and against the anvil. When the tissue has thus been fastened, the actuator is operated to retract the-now empty fastener holder, thereby releasing the tissue from the instrument. Linear fastener or stapling instruments of this type are disclosed in commonly assigned U.S. Pat. Nos. 4,354,628, 4,383,634, 4,402,444, 4,506,671, 4,530,453, 4,573,622, 4,728,020 and 5,100,042, the contents of each being incorporated herein by reference.

In the instruments of the type aforedescribed, the fastener holder and anvil components are generally aligned with respect to the axis of the instrument. However, in certain applications it may be desirable for the fastener holder and anvil components to be offset, which would thereby facilitate access to remote tissue areas and enhance visibility during the clamping procedure.

### SUMMARY OF THE INVENTION

Accordingly, the present disclosure is directed to a surgical fastening apparatus which incorporates a fastener holder and anvil component offset relative to the axis of the instrument at a predetermined angular orientation(s). Generally stated, the present invention is directed to a surgical fastener instrument including a handle, an elongated member extending distally from the handle and a fastener applier and anvil head disposed at the distal end of the elongated member. The elongated member defines a distal offset end upon which the fastener applier and the anvil head are mounted. The offset end is angularly oriented with respect to the axis of the instrument at a predetermined fixed angle (e.g., either 45°, 60° or 75° relative to the axis of the instrument) thereby positioning the fastener applier and the anvil head at the fixed angle. This feature facilitates positioning of the tissue with respect to the instrument and enhances visual access to the tissue when appropriately disposed within the fastener applier and the anvil head. Various mechanisms for effectuating the clamping of the tissue between the fastener holder and anvil, and the firing of the instrument are disclosed.

### BRIEF DESCRIPTION OF THE DRAWINGS

The foregoing features of the present disclosure will become more readily apparent and will be better understood by referring to the following detailed description of preferred embodiments, which are described hereinbelow with reference to the drawings wherein:

FIG. 1 is a perspective view of the surgical fastener applying apparatus in accordance with the principles of the present invention;

FIG. 2 is a top plan view of the apparatus of FIG. 1;

FIG. 3 is a side view in partial cross-section of the apparatus illustrating components of the clamping and fastener firing mechanisms;

FIGS. 4-5 are views similar to the view of FIG. 2 illustrating successive stages in the operating cycle of the apparatus;

FIGS. 6-9 are partly sectional elevational views of the fastener firing and anvil head illustrating the operating stages of the apparatus;

FIG. 10 is a drawing illustrating the arrangement of the firing and approximating bands within the elongated member of the apparatus;

FIG. 10A is a cross-sectional view taken along the lines 10A-10A of FIG. 10;

FIGS. 11-18 are drawings illustrating alternate embodiments of the apparatus; and

FIGS. 19-20 are perspective and top plan views, respectively, of another embodiment of the present invention featuring a fastener firing and anvil head parallel to the axis of the instrument.

### DETAILED DESCRIPTION OF PREFERRED EMBODIMENTS

Although the principles of the present invention are applicable to a variety of surgical fastener apparatti, the following discussion will focus on a surgical fastener instrument having application in a thoracic-abdominal surgical procedure and being adapted to simultaneously fire a plurality of fasteners across tissue, e.g., through hollow body organs such as the thorax, trachea, stomach or intestines. More specifically, the fastener instrument will be described in terms of firing a two-part surgical fastener of the type aforedescribed. However, it is appreciated that the present application is suitable for any surgical fastener apparatti including clip or single staple applier instruments, etc.

In the following description, as traditional, the term “proximal” will refer to the portion of the instrument closest to the operator while the term “distal” refers to the portion of the instrument most remote from the operator.

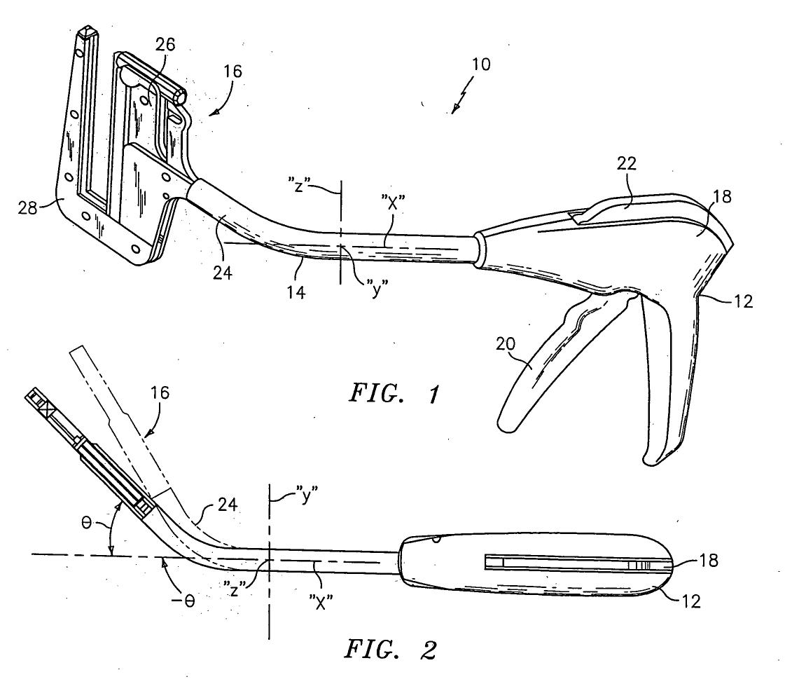

With initial reference to FIGS. 1-2, fastener instrument 10 generally includes handle 12, elongated member 14 extending distally from the handle 12 and fastener applier and anvil head 16 connected to the distal end of the elongated member 14. Handle 12 defines a pistol-grip configuration consisting of frame 18 and fastener firing lever 20 depending from the frame 18. Firing lever 20 is dimensioned for engagement by the fingers of the operator. Lever 20 is a component of the fastener firing mechanism. Handle 12 further includes clamp actuator 22 which is pivotally mounted to the upper surface of the frame 18. Clamp actuator 22 is a component of an approximating mechanism. The fastener firing mechanism and approximating mechanism will be discussed in detail hereinbelow.

Elongated member 14 defines longitudinal axis “x” extending the length of the elongated member 14, lateral axis “y” and transverse axis “z”. Elongated member 14 further includes an offset distal end 24. Offset end 24 is angularly displaced in the direction of lateral axis “y” at a predetermined angle “θ”. This offset feature positions fastener firing and anvil head 16 in angular relationship with respect to longitudinal axis “x”. Such arrangement facilitates positioning of fastener applier and anvil head 16 relative to the tissue or organ to be treated in remote or tortuous locations. Furthermore, in certain situations, viewing of the positioning of the tissue within the fastener applier and anvil head 16 is enhanced by virtue of the consequent offset or angled relationship of the assembly. The predetermined angle “θ” may range between 0°-90°. In a preferred arrangement, angle “θ” may be about either 45°, 60° or 75° relative to the longitudinal axis “x”. It is further appreciated that offset distal end 24 of elongated member 14 may be offset in an opposite direction to that depicted in the figures, i.e., at a negative angle “θ” shown in FIG. 2. In addition, offset end 24 may extend from the remaining portion of elongated member 14 at a sharper angle rather than the arcuate arrangement shown in the figures. It is further envisioned that the offset end may extend along the “z” axis or have a direction with both a “y” component and a “z” component.

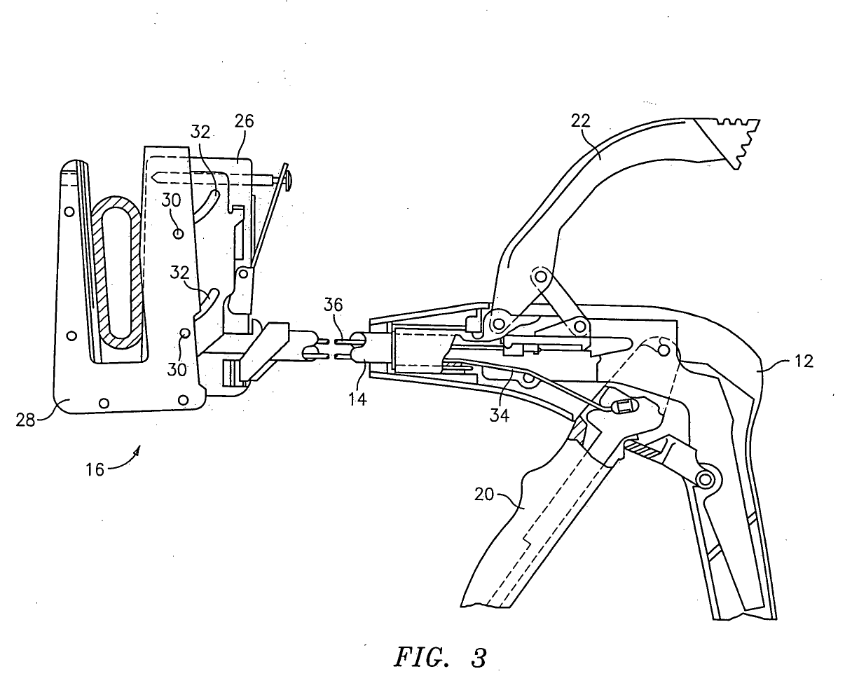

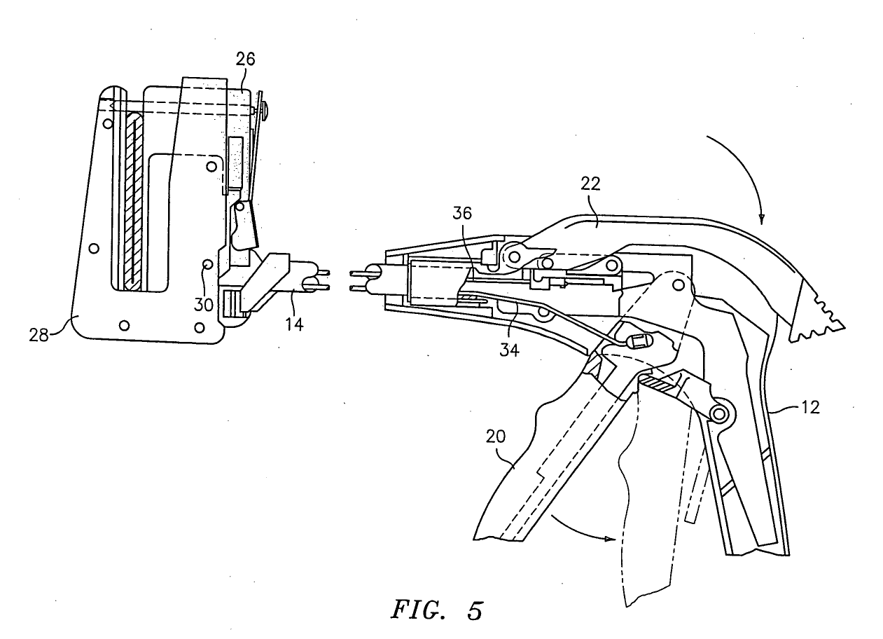

Referring now to FIGS. 3-5, in conjunction with FIGS. 1-2, fastener applier and anvil head 16 will be discussed. Fastener applier and anvil head 16 include proximal fastener holder 26 and U-shaped frame 28 which is mounted for movement relative to the fastener holder 26. Fastener holder 26 accommodates a plurality of fasteners which are driven into the tissue upon actuation of firing lever 20. U-shaped frame 28 includes a plurality of mounting pins 30 which are received within corresponding cam slots 32 of fastener holder 26 to operatively connect the components. U-shaped frame 28 is adapted to move between an open position depicted in FIG. 3 to a closed position depicted in FIG. 5 upon actuation of the approximating mechanism. U-shaped frame 28 also houses a plurality of retainer parts of the two-part fastener, which receives the legs of the fasteners in fastener holder 26.

Firing lever 20 is operatively connected to one or more flexible bands 34 which extend the length of elongated member 14 preferably within a central lumen of the elongated member 14. Similarly, clamp actuator 22 is connected to one or more clamping bands 36 which extend in a pair of corresponding peripheral lumens of elongated member 14. Firing bands 34 and clamping bands 36 may be connected to respective firing lever 20 and clamp actuator 22 through conventional means as appreciated by one skilled in the art. Firing bands 34 may be fabricated from any suitable material having sufficient rigidity or strength in tension to actuate the firing assembly while permitting the firing band 34 to bend along the angled path defined by the offset portion 24 of the elongated member 14. One skilled in the art may appreciate various materials for firing band 34 including soft metal, shape-memory metal, polymeric material, etc. It is also envisioned that firing band 34 may be a substantially solid guide wire material with a plurality of cuts on one side adjacent the bend in the elongated member 14. The cuts permit bending or flexing of the guide wire in the direction of the cuts. Other methodologies are also envisioned. Clamping bands 36 may be a laminated band or the like having sufficient strength in tension to effectuate movement of the U-shaped frame 28 upon actuation of the approximating mechanism. Other suitable materials are also envisioned.

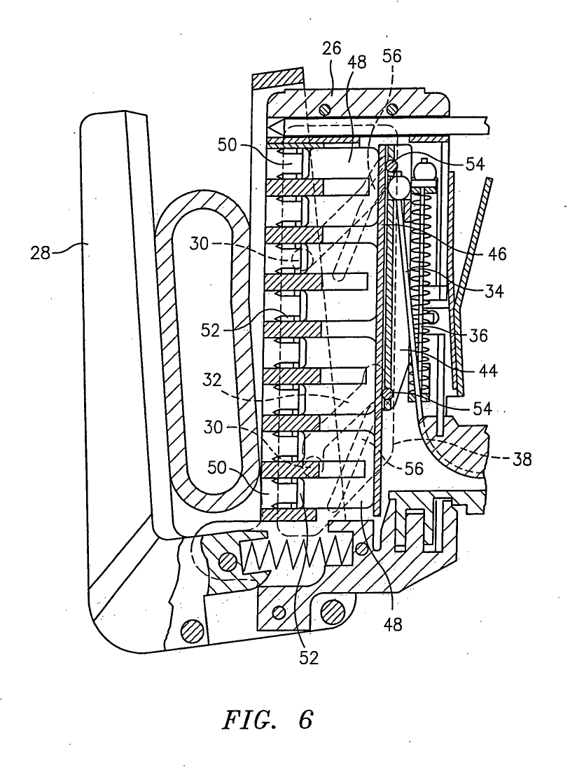

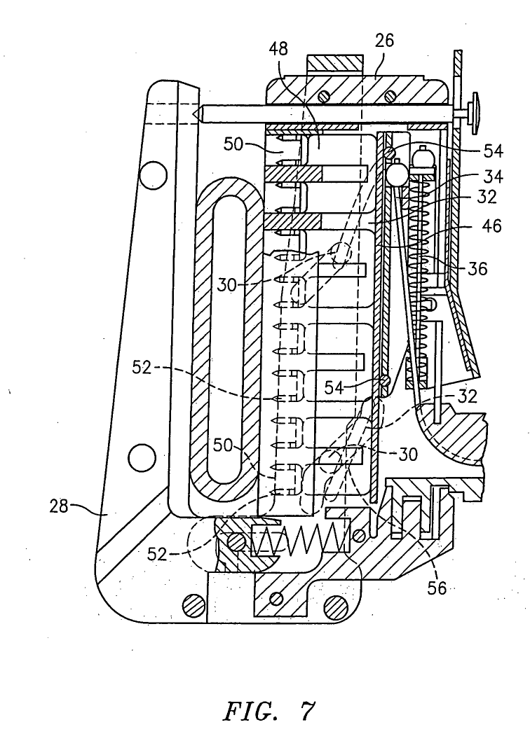

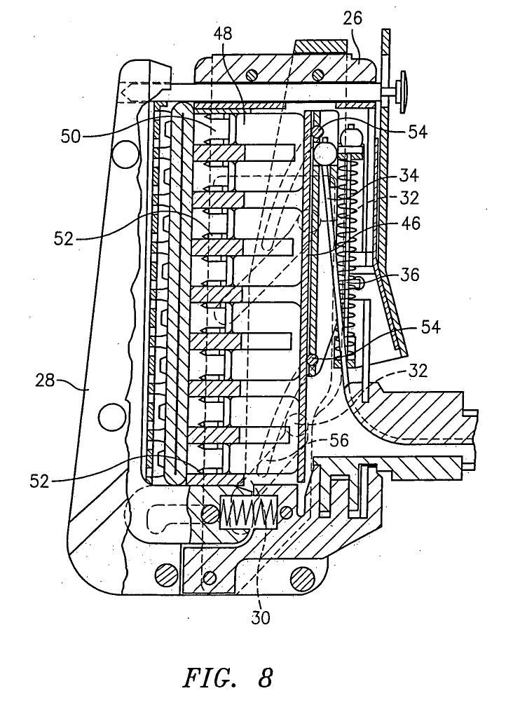

With reference to FIG. 6, in connection with FIGS. 3-5, clamping bands 36 are operatively connected to U-shaped frame 28 through a camming arrangement. The camming arrangement includes cam plates 38 (shown in phantom), cam followers 30 which project into cam slots 32 disposed in fastener holder 26. When clamp actuator 22 is depressed, clamping bands 36 are pulled in the proximal direction, which pulls cam plates 38 vertically down relative to fastener holder 26 as shown progressively in FIGS. 6-8. This movement causes U-shaped frame 28 to pivot and move toward fastener holder 26 through cooperation of components 40, 42 to the position depicted in FIG. 8. This camming arrangement is disclosed in the Green '020 patent which is already incorporated herein by reference. Other arrangements are also envisioned and may be readily appreciated by one skilled in the art.

With reference now to FIGS. 3-6, firing bands 34 are operatively connected to pusher actuator 44 disposed within fastener holder 26 of the fastener applier and anvil head 16. Pusher actuator 44 is engageable with rigid pusher follower 46 which is arranged to contact a plurality of pusher fingers 48. Pusher fingers 48 extend into the rear of respective apertures 50 which houses a respective fastener part 52 of the two-part fasteners. Pusher actuator 44 is supported by cam follower pins 54 which extend into cam slots 56 of fastener holder 26. When firing lever 20 is operated to pull firing bands 34 in the proximal direction, the distal ends of the bands 34 pull down on pusher actuator 44. As pusher actuator 44 moves down, it is also forced to move in the distal direction by cooperation of elements 54, 56. The distal motion of pusher actuator 44 is imparted to pusher fingers 48 via pusher follower 46 thereby driving fastener parts 52 partly through tissue and into interlocking engagement with retainer parts to fasten the tissue. Further specifics of the components of fastener firing mechanism may be ascertained by reference to the Green '020 patent which is previously incorporated herein by reference.

The operation of the approximating and fastener firing mechanisms will now be discussed. The surgical instrument 10 is positioned at the target tissue location with the tissue disposed within the U-shaped frame 28 of fastener applier and anvil head 16. As discussed, the offset arrangement of the fastener firing assembly may assist in both positioning and visualizing of the tissue relative to the instrument. Thereafter, clamp actuator 22 is pivoted in a downward direction relative to frame to thereby cause clamping bands 36 to move in a proximal direction. Proximal movement of the clamping bands 36 causes the U-shaped frame 28 to move, as effectuated via the camming arrangement, from the position depicted in FIG. 6 to the position depicted in FIG. 8, securely clamping the tissue therebetween as discussed hereinabove.

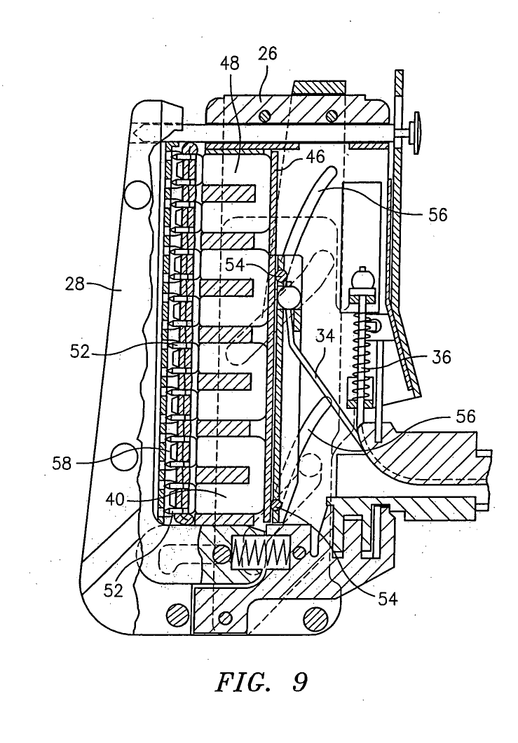

With reference to FIG. 9, once the tissue is clamped, attention is directed to firing the fastener firing mechanism. When firing lever 20 is operated to pull firing bands 34 in the proximal direction, the distal end(s) of the bands pull down on pusher actuator 44. As pusher actuator 44 moves downwardly, a distal motion is imparted to pusher fingers 48 via pusher follower 46 thereby driving fastener parts 52 partly through tissue and into interlocking engagement with the corresponding retainer parts 58 within U-shaped frame 28 to fasten tissue.

FIGS. 10 and 10A are views of the arrangement of firing and clamping bands 34, 36 within elongated member 14 in connection with the embodiment of FIGS. 1-9.

Use of the angled fastener firing instrument provides significant advantages over conventional linear or straight fastener appliers. By virtue of the angled fastener firing assembly, access to various remote tissue sites may be readily achieved. In addition, visual access to the object tissue within fastener firing assembly is enhanced.

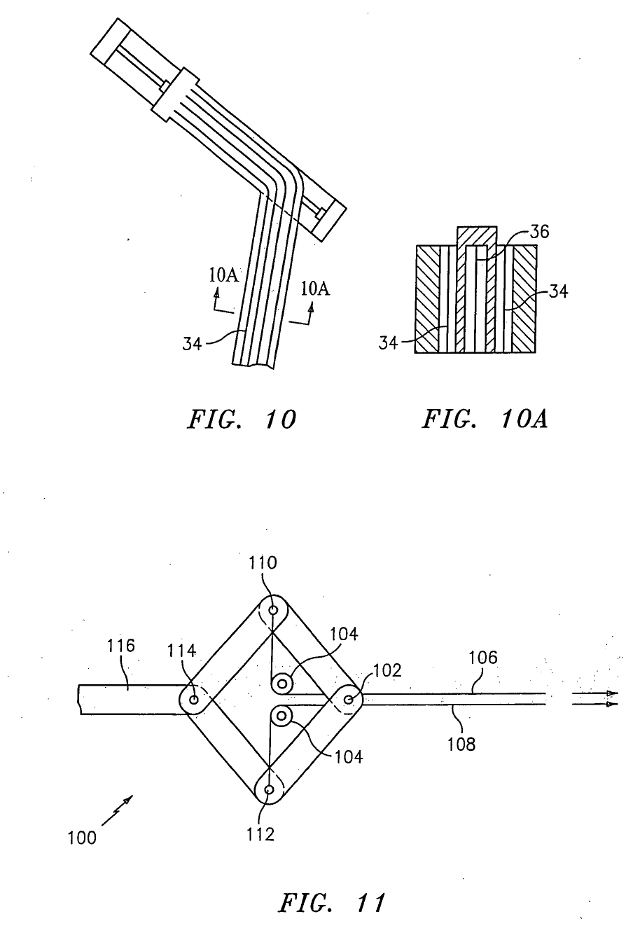

FIGS. 11-18 illustrate alternate arrangements for actuating the approximating and firing mechanisms of the fixed angle instrument of FIG. 1. These arrangements will be discussed generally with respect to their interaction with the pusher actuator of the firing mechanism and/or camming arrangement of the approximating mechanism of the apparatus disclosed in FIGS. 1-10; however, it is appreciated that the various alternate arrangements to be described may be adapted with any fastener firing or approximating mechanism as appreciated by one skilled in the art.

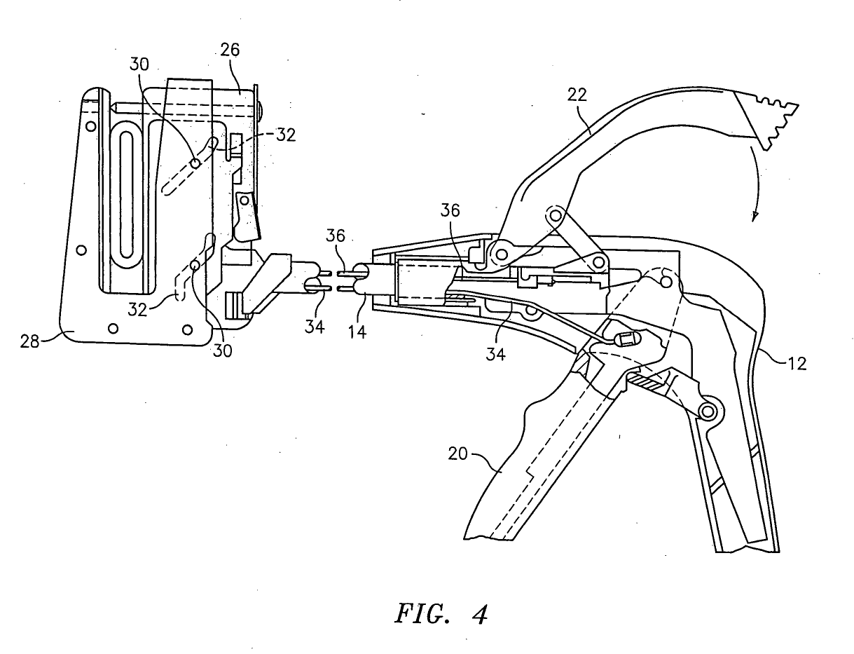

In FIG. 11, a “scissor jack” type toggle is disclosed. This arrangement includes a linkage 100 having a fixed pin 102 at its proximal end and a pair of pulleys 104 disposed in the center of the linkage. Cables 106, 108 are connected to respective intermediate connecting pins 110, 112 between adjacent links, and slide along the pulleys 104 during movement of the cables. Cables 106, 108 may be connected to firing lever 20 and/or clamp actuator 22. The distal end or pin 114 of the linkage is connected to, depending on its use, the pusher actuator 44 of the fastener firing mechanism and/or the camming arrangement of the approximating mechanism. Upon actuation, the cables 106, 108 move proximally to cause the linkage to actuate to thereby move pin 114 in the proximal direction. Proximal movement of the pin 114 causes actuation of the approximating or fastener firing mechanisms to effect either approximation of U-shaped frame 28 and/or firing of the fasteners. It is envisioned that this scissor jack embodiment may be incorporated into the fastener applier and anvil head 16 on offset end 24 of the elongated member 14. For example, with regard to the approximation mechanism, pin 114 may be connected by conventional means, e.g., to band 166 which, in turn, is a component or connected to the camming arrangement depicted in FIG. 4. As discussed hereinabove, the camming arrangement is inclusive of cam plates 38, cam followers, 30 and cam slots 32. Accordingly, actuation of the approximating mechanism will cause proximal movement of cables 106, 108 and movement of the cam plates 38 downwardly thereby causing pivoting movement of the U-shaped frame to a firing position in the same general manner aforedescribed in connection with FIG. 6. With regard to the firing mechanism, pin 114 of the scissor-jack may be connected to pusher actuator 44 of the fastener applier and anvil head through bands 16. Band 116 may be integral with pusher actuator 44 or a separate element connected thereto. Thus, actuation of the firing lever 20 causes proximal movement of pin 114 and band 116 to thereby actuate pusher actuator 44 to drive the staples in the manner aforedescribed in connection with FIG. 6. It is further envisioned that two “scissor jack” arrangements may be utilized, one as a component of the approximating mechanism and the other as a component of the firing mechanism.

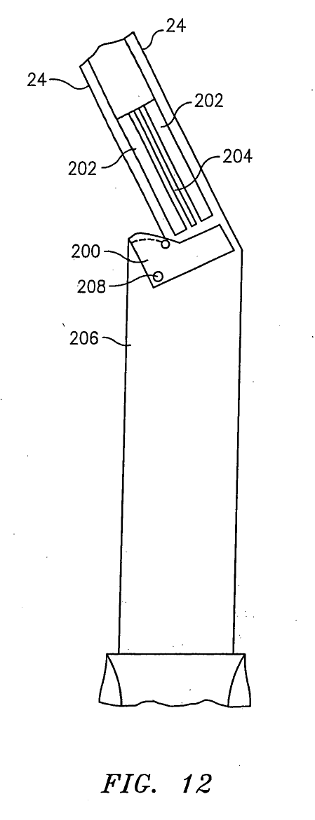

FIG. 12 illustrates another arrangement for incorporation into the apparatus of the present invention. A crank mechanism 200 may be positioned adjacent the bend of the offset distal end 24 of elongated member 14. A pair of clamp bars 202 and pusher bar 204 extend within the offset distal end 24 to operatively engage the camming arrangement of the approximating mechanism and the pusher actuator 44 of the firing mechanism. A cable 206 extends from the crank 200 to the handle and is operatively engageable with the clamp actuator 22 and firing lever 30. Actuation of the mechanisms causes the cable 206 to move proximally thereby pivoting crank 200 about pivot pin 208 to cause advancing movement of the clamp bars 202 and staple pusher bar 204. Such advancing movement approximates the U-shaped frame 28 and fires the fasteners from the fastener holder 26. One skilled in the art may readily adapt this arrangement in the fastener firing head to achieve the objectives herein. It is noted that the handle of the instrument may be adapted to provide a single lever to actuate the cable to cause the corresponding actuation of the approximating and firing assemblies. Such arrangement is disclosed in commonly assigned application Ser. No. 09/687,815, filed Oct. 17, 2000, the contents of which are incorporated herein by reference.

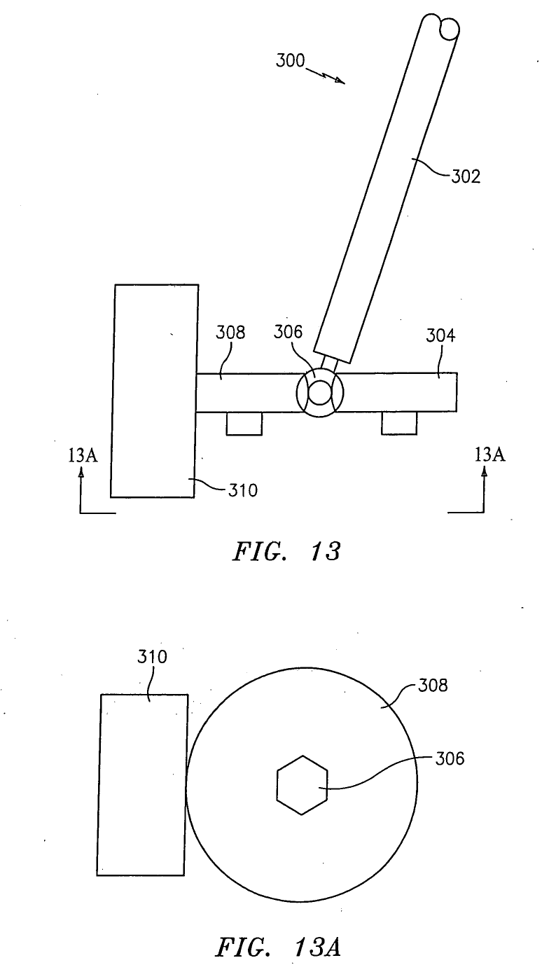

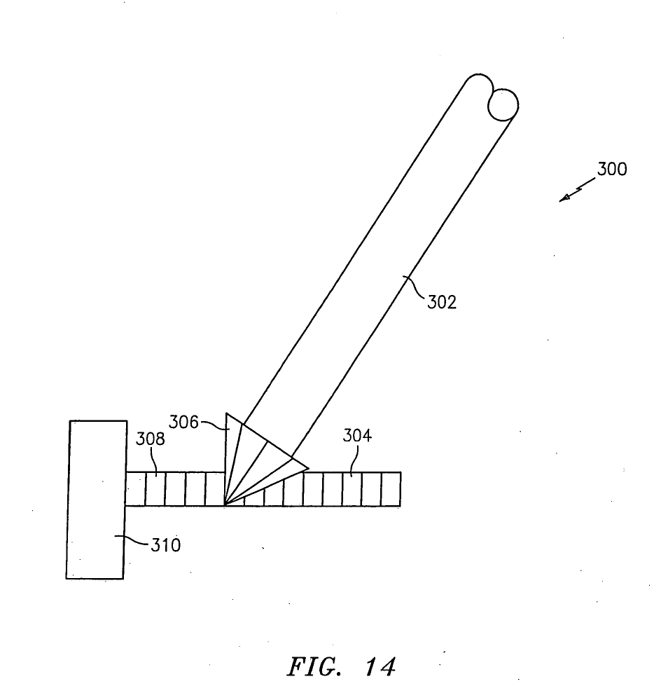

FIGS. 13-14 illustrate another alternate embodiment including a ball drive arrangement 300 having a driver shaft 302. Drive shaft 302 is connected at its proximal end to the firing lever 20 of the handle and at its distal end to an eccentric bearing 304. The driver shaft 302 defines a hex or ball drive 306 at its distal end which engages the eccentric bearing 304. On its other side, the ball drive 306 is engageable with a second hex drive 308. A pusher 310 preferably extends within the offset end 24 of the elongated member 14 and is operatively engageable with the pusher actuator 44 of the firing mechanism and/or camming arrangement of the approximating mechanism. The pusher 310 has teeth which form part of a rack and pinion arrangement. Rotation of the driver shaft 302 causes the eccentric bearing 304 (which has teeth engageable with the ball drive 306) to rotate. As a consequence, the eccentric bearing 304 urges the second hex drive 308 into engagement with the pusher 310. Further rotation causes the pusher 310 to translate through the rack and pinion arrangement to actuate the firing mechanism and/or approximating mechanism. As appreciated, the drive shaft 302 is connected at its proximal end to the manual actuator(s) of the approximating and/or firing mechanism.

FIG. 15 illustrates a clamp mechanism 400 which may be utilized with the fixed angle instrument of the present invention. A cam arrangement 402 consisting of a plurality of cam slots, levers and pins is provided to actuate the approximating mechanism. Actuation of the approximating mechanism is effected by pulling the cable in the proximal direction which causes the cam arrangement to move in the manner described in the drawing thus closing u-shaped frame 28 for subsequent firing of the fasteners.

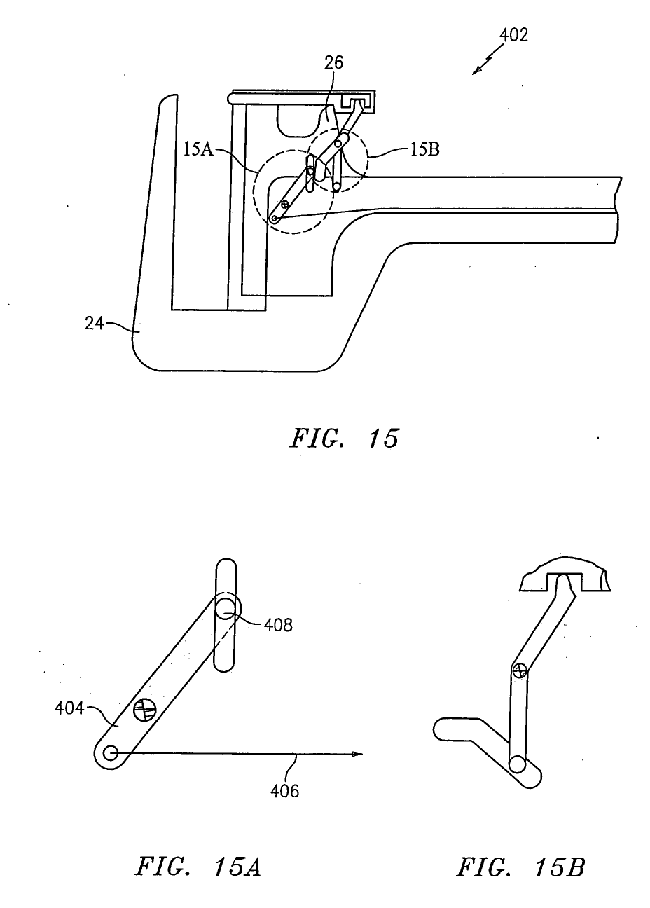

FIG. 16 illustrates another alternate embodiment. With this embodiment, a clamping cable 600 and firing cable 602 extend the length of the elongated member 14 in operative engagement with a single actuator which actuates the approximating and firing mechanisms. The cables 600, 602 are looped about a fixed rivet bushing 604 in U-shaped frame 28. The extreme distal end of the firing cable 602 is operatively connected to the pusher actuator 44 of the firing mechanism while the extreme end of the approximating cable 600 is fixed to the fastener holder 26. Actuation of the handle causes the firing cable 602 and clamping cable 600 to move proximally. Proximal movement of the clamping cable 600 closes or approximates the U-shaped frame 28 with respect to fastener holder 26. Proximal movement of the firing cable 602 actuates pusher actuator 44 to fire the fasteners.

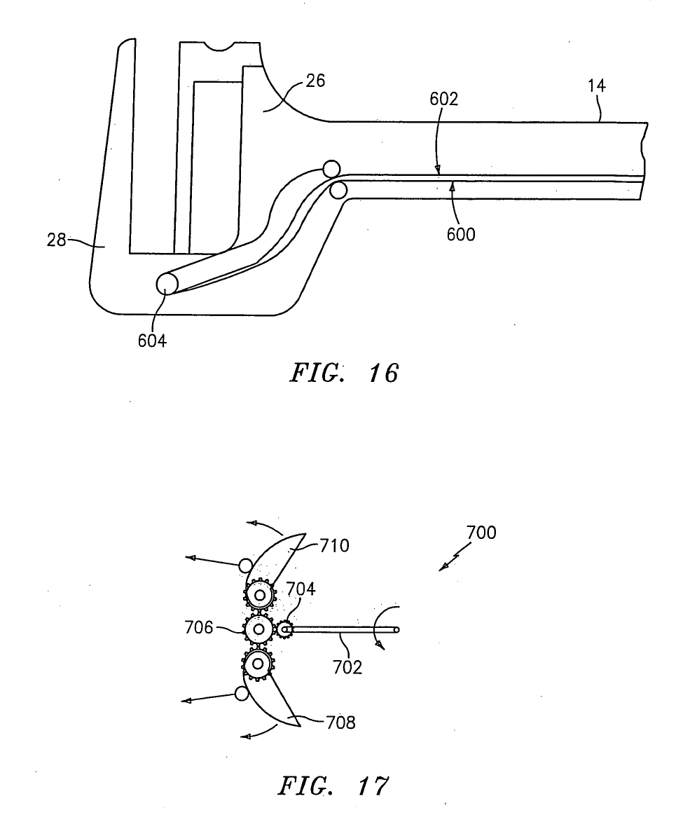

FIG. 17 illustrates another embodiment incorporating a gear/cam combination 700. A single drive 702 extends to the handle and is connected to a single actuator for actuating the approximating and firing mechanisms. The drive 702 includes gear 704 at its distal end, which cooperates with a central gear 706 of the combination to actuate 1) a first cam 708 which is engageable with the approximating cam to actuate he approximating mechanism and 2) a second cam 710 engageable with the pusher actuator 44 to actuate the firing mechanism.

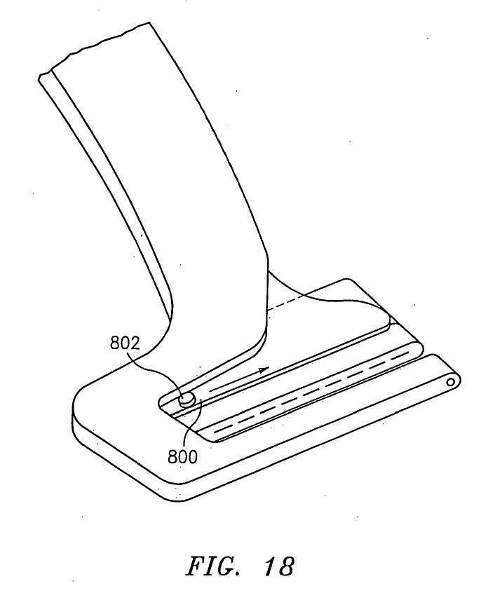

FIG. 18 illustrates another embodiment incorporating a built-in knife 800 which traverses the opening in the U-shaped frame 28 to sever the tissue subsequent to firing an array of staples. The knife includes a finger tab 802 for manual operation.

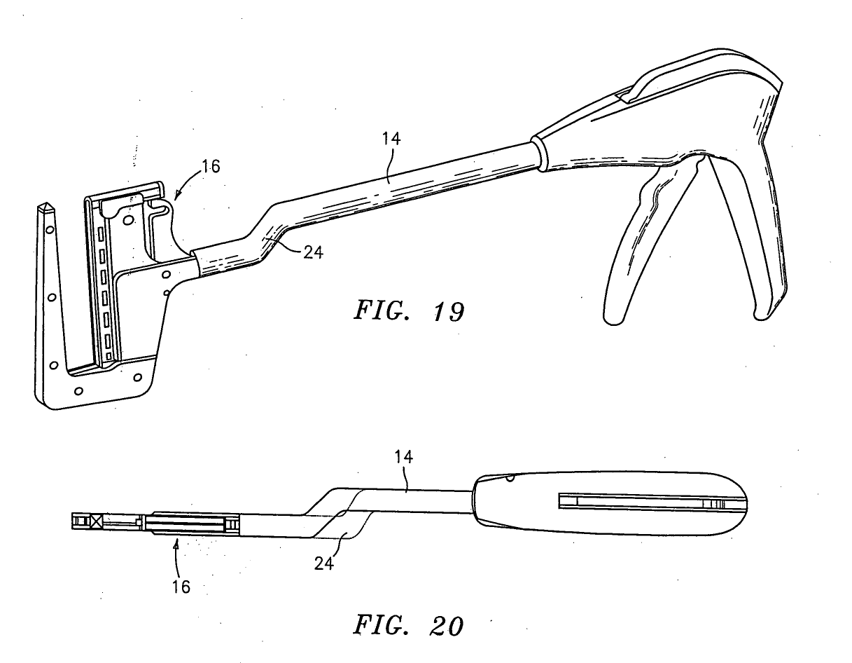

FIGS. 19-20 illustrate another embodiment in which the offset distal end 24 is offset but in parallel relation to the axis “y” of the instrument. This arrangement may prove useful in certain situations where direct linear access to the tissue site is inhibited. Any of the aforedescribed firing or approximating mechanisms may be incorporated into this instrument.

While the invention has been particularly shown, and described with reference to the preferred embodiments, it will be understood by those skilled in the art that various modifications and changes in form and detail may be made therein without departing from the scope and spirit of the invention. Accordingly, modifications such as those suggested above, but not limited thereto, are to be considered within the scope of the invention.

We claim:

1. A surgical fastener instrument comprising:
a handle;
an elongated member extending distally from the handle, the elongated member defining a longitudinal axis extending the length of the elongated member, the elongated member having an offset distal end portion angularly arranged at a predetermined angle with respect to the longitudinal axis; and
a fastener applying mechanism mounted to the offset distal end portion of the elongated member, the fastener applying mechanism having at least one fastener adapted to be driven into tissue.

2. The surgical fastener instrument of claim 1 including a proximal actuator mounted to the handle and operatively connected to the fastener applying mechanism, the actuator movable relative to the handle to actuate the fastener applying mechanism.

3. The surgical fastener instrument of claim 2 wherein the proximal actuator includes a fastener firing lever depending from the frame to define a pistol grip configuration, the firing lever being dimensioned for engagement by the fingers of the operator.

4. The surgical fastener instrument of claim 2 wherein the fastener applying mechanism includes a fastener holder and an anvil, the fastener holder and the anvil adapter for relative movement between an open position and an approximated position.

5. The surgical fastener instrument of claim 4 including an approximating mechanism for moving the fastener holder and the anvil between the open position and an approximated position.

6. The surgical fastener instrument of claim 5 wherein the fastener applying mechanism includes a flexible cable extending within the elongated member and operatively connected to the proximal actuator.

7. The surgical fastener instrument of claim 5 including a proximal approximator mounted to the handle and operatively connected to the approximating mechanism, the approximator movable relative to the handle to actuate the approximating mechanism.

8. The surgical fastener instrument of claim 7 wherein the approximating mechanism includes a flexible cable extending within the elongated member and operatively connected to the proximal approximator.

9. The surgical fastener instrument of claim 7 wherein the fastener holder and the anvil define a general U-shaped structure, the anvil being on a distal leg of the U-shaped structure and the fastener holder being on the proximal leg of the U-shaped structure, the anvil and the fastener holder being adapted for relative movement to clamp tissue to be fastened between the fastener holding part and the anvil part when in the approximated position.

10. The surgical fastener instrument of claim 9 wherein at least one of the fastener applying mechanism and the approximating mechanism includes a “scissor jack” type toggle linkage, the “scissor jack” type toggle linkage being operatively connected to the respective proximal actuator or proximal approximator.

11. The surgical fastener instrument of claim 9 wherein at least one of the fastener applying mechanism and the approximating mechanism includes a crank mechanism, the crank mechanism further including a cam arrangement, said cam arrangement further includes a plurality of cam slots, levers, and pins to actuate the respective mechanism.

12. The surgical fastener instrument of claim 9 wherein at least one of the fastener applying mechanism and the approximating mechanism includes a ball drive arrangement, the ball drive arrangement including an eccentric bearing and a hex or ball drive engageable with the eccentric bearing, the bearing rotatable to drive a-pusher to actuate the respective fastener applying or approximating mechanism.

13. The surgical fastener instrument of claim 9 wherein at least one of the fastener applying mechanism and the approximating mechanism includes a gear arrangement having a plurality of gears, the gear cooperating to actuate the respective fastener applying or approximating mechanisms.

14. The surgical fastener instrument of claim 1 wherein the predetermined angle of the offset distal end of elongated member is in the range from about 0° to about 90°.

15. The surgical fastener instrument of claim 14 wherein the predetermined angle is about 45°.

16. The surgical fastener instrument of claim 14 wherein the predetermined angle is about 60°.

17. The surgical fastener instrument of claim 14 wherein the predetermined angle is about 75°.

18. The surgical fastener instrument of claim 3 wherein the fastener applying mechanism includes a knife, the knife further including a finger tab for manual operation.

19. A surgical fastener instrument comprising:
a handle;
an elongated member extending distally from the handle, the elongated member defining a longitudinal axis extending the length of the elongated member, the elongated member having an offset distal end wherein said offset distal end is in parallel relation to the lateral axis of the instrument;
a fastener applying mechanism mounted to the offset distal end of the elongated member, the fastener applying mechanism having at least one fastener; and
a proximal actuator assembly including means for firing at least one fastener.
